(19) United States
(12) Patent Application Publication
(10) Pub. No.: US 2005/0009789 A1
(43) Pub. Date: Jan. 13, 2005

(54) Cyclooxygenase inhibition with nitroxyl

(75) Inventors: David A. Wink, Hagerstown, MD (US); Katrina M. Miranda, Tucson, AZ (US); Christopher M. Bradbury, Burlington, VT (US); David Gius, Clarksville, MD (US); Jon M. Fukuto, Agoura, CA (US); Martin Feelisch, Needham, MA (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: The Government of the USA as represented by the Secretary of the Dept. of Health and Human Service

(21) Appl. No.: 10/845,619

(22) Filed: May 12, 2004

Related U.S. Application Data
(60) Provisional application No. 60/470,320, filed on May 13, 2003.

(51) Int. Cl. A61K 31/655

(52) U.S. Cl. 514/149

(57) ABSTRACT

Nitroxyl is used to inhibit COX-2 activity and particularly to selectively inhibit COX-2 activity. Nitroxyl also is used to treat conditions that respond favorably to inhibition of COX-2 activity in subjects having such conditions. In some cases nitroxyl is used to treat conditions that respond favorably to inhibition of COX-2 activity in subjects having such conditions and who also have at least one other condition for which inhibition of COX-1 activity is disadvantageous.

Nitroxyl can be provided directly, but typically is provided with the use of a nitroxyl donor. Nitroxyl donors include any agent or compound (or combination thereof) that donates HNO or NO⁻. Diazeniumdiolates are used in some cases as nitroxyl donors. In particular instances, diazeniumdiolates having a primary amine group are used as nitroxyl donors. Nitroxyl-donating compounds also are screened for selective COX-2 inhibition for identification as therapeutic agents.

### CROSS REFERENCE TO RELATED APPLICATION

This disclosure claims the benefit of U.S. Provisional Patent Application No. 60/470,320, filed May 13, 2003, which is incorporated by reference herein.

### FIELD

The methods disclosed herein relate to the use of pharmaceutical compounds to inhibit cyclooxygenase-2 activity, to treat cyclooxygenase-2 mediated conditions, as well as to screening compounds for such activity.

### BACKGROUND

Prostaglandins play a critical role in the pathophysiology of inflammation. In particular, inflammation is initiated and maintained by the overproduction of prostaglandins in injured cells. Prostaglandins are biosynthesized on demand from arachidonic acid, a 20-carbon fatty acid that is derived from the breakdown of cell-membrane phospholipids. The first step in the synthesis of prostaglandins occurs when the enzyme cyclooxygenase (COX) (also known as prostaglandin H synthase (PGHS)) catalyzes the conversion of arachidonic acid into the endoperoxide PGG₂ and then into PGH₂. PGH₂ is in turn metabolized by one or more prostaglandin synthases (PGE₂ synthase, PGD₂ synthase, etc.) to generate the final “2-series” prostaglandins, such as PGE₂, PGD₂, PGF₂, PGI₂, as well as thromboxanes and prostacyclins.

As disclosed in U.S. Pat. No. 6,048,850 to Young et al., there are two forms of COX. Cyclooxygenase-1 (COX-1) is constitutively expressed in most tissues. It is a “housekeeping” enzyme that regulates normal cellular processes, such as gastric cytoprotection, vascular homeostasis, platelet aggregation, and kidney function.

Cyclooxygenase-2 (COX-2) is usually undetectable in most tissues. However, its expression is increased during states of inflammation or in response to mitogenic stimuli. COX-2 is accordingly referred to as “inducible.” This inducible COX-2 is responsible for prostaglandin overproduction through the COX pathway in response to tissue injury and stimulation by growth factors and proinflammatory cytokines.

As the rate-limiting step for prostaglandin synthesis, the COX pathway is the principal target for anti-inflammatory drug action. Inhibition of COX activity accounts for the activity of the non-steroidal anti-inflammatory drugs (NSAIDs), such as aspirin, acetaminophen, ibuprofen, naproxen, indomethacin. Unfortunately, these drugs are nonselective COX inhibitors. Thus, they inhibit the activity of COX-2 in inflammation, which produces a desirable therapeutic effect. But they also significantly inhibit the activity of COX-1 in non-inflamed cells, which interferes with the normal production of prostaglandins necessary for “housecleaning” functions. COX-1 inhibition can produce undesirable side effects, such as renal failure, and gastrointestinal mucosal disorders, for example, gastritis, gastrointestinal bleeding, and ulcers. An estimated 16,500 deaths each year result from gastrointestinal bleeding associated with traditional NSAIDs. Moskowitz, Consultant, 40:1370 (2000).

COX-2 selectivity can be quantified by calculating the COX-2/COX-1 IC₅₀ (inhibitor concentration at which 50% inhibition occurs) ratio. Compounds with a ratio less than one can be considered COX-2 selective. The lower the COX-2/COX-1 IC₅₀ ratio, the higher the COX-2 selectivity.

COX-2 inhibiting compounds have been reported to be useful in treating a variety of conditions mediated, at least in part, by inflammation. For example, COX-2 inhibitors have been suggested to treat conditions such as general pain, osteoarthritis and rheumatoid arthritis, see Whelton et al., Am. J. Ther., 7(3):159-75 (2000), menstrual pain associated with primary dysmenorrhea, see Daniels et al., Obstet. Gynecol., 100(2):350-8 (2002), cancers, such as colon cancer, see Nagatsuka, et al., Int'l. J Cancer, 100(5):515-9 (2002), oral cancer, see Wang et al., Laryngoscope, 112(5):839-43 (2002), and skin cancer, see Lee et al., Anticancer Res., 22(4):2089-96 (2002); Fischer, J. Environ. Pathol. Toxicol. Oncol. 21(2):183-91(2002), Alzheimer's disease, see Aisen, J. Pain Symptom Manage., 23(4 Suppl):S35-40 (2002), and diabetes (insulin dependent diabetes mellitus in particular), see Tabatabaie et al., Biochem Biophys. Res. Commun., 273(2):699-704 (2000).

### SUMMARY

Nitroxyl has been found to inhibit COX-2 activity. In particular, nitroxyl selectively inhibits COX-2 activity. In some cases the COX-2/COX-1 IC₅₀ ratio of nitroxyl is about 0.25 or less, for example, from about 0.2 to about 0.01. Also, COX inhibition by nitroxyl is dose dependent with the dose response curve for COX-2 inhibition being significantly steeper than the dose response curve for COX-1 inhibition.

Methods of using nitroxyl to inhibit COX-2 activity, and particularly to selectively inhibit COX-2 activity, are disclosed herein. Also disclosed are methods of using nitroxyl to treat conditions that respond favorably to COX-2 inhibition in subjects having such conditions. In some cases nitroxyl is used to treat conditions that respond favorably to COX-2 inhibition in subjects having such conditions and who also have at least one other condition for which COX-1 inhibition is disadvantageous.

Typically, one or more nitroxyl-donating compounds are used to provide nitroxyl to inhibit COX-2. Any physiologically acceptable nitroxyl-donating compound can be used. Such compounds include, but are not limited to, nitroxyl-donating diazeniumdiolates (J-N(O)NO) and their salts. For example, Angeli's salt (Na₂ON(O)NO) is used to donate nitroxyl in some instances. In particular cases, nitroxyl-donating diazeniumdiolates having a primary amine group attached to the NONO group (J=RNH) are used to donate nitroxyl. For example, IPA/NO (Na(CH₃)₂C(H)N(H)N(O)NO) or derivatives or analogs thereof, or combinations thereof, are used to donate nitroxyl in some instances. Alternatively, other nitroxyl donors are used, such as hydroxamic acids and their salts (for example, Piloty's acid).

Methods of screening candidate compounds for COX-2 inhibition (including selective COX-2 inhibition) also are disclosed herein. In some cases screening is accomplished by enzyme immuno assay.

### BRIEF DESCRIPTION OF THE FIGURES

FIG. 1 is a graph showing the COX-1 and COX-2 inhibition in COX-1 and COX-2 systems caused by the nitroxyl donor Angeli's salt at concentrations from 0.001 μM to 1000 μM.

FIG. 2 is a graph showing the COX-1 and COX-2 inhibition in different COX-1 and COX-2 systems from those shown in FIG. 1 caused by the nitroxyl donor Angeli's salt at concentrations from 0.001 μM to 1000 PM.

FIG. 3 is a graph showing the COX-2 inhibition in COX-2 systems caused by the nitroxyl donors Angeli's salt and IPA/NO at concentrations from 25 μM to 1000 μM.

### DETAILED DESCRIPTION

A “subject” is an animal, such as a mammal, for example, a human.

“Nitroxyl” is HNO/NO⁻.

“NO” is the free radical nitric oxide.

A “nitroxyl donor” is an agent or compound (or combination of agents or compounds) that donates HNO or NO⁻. Further, when referring to nitroxyl donating compounds herein, salts of such compounds are also included.

A “candidate compound” is a compound that is known to donate nitroxyl or has a chemical structure similar to a known nitroxyl donor. Knowledge as to whether the candidate compound is a nitroxyl donor can be, for example, from the literature or from testing the candidate compound for nitroxyl donation.

“Nitroxyl donation pH” is the pH at which and above which a nitroxyl-donating compounds donates nitroxyl.

“Selective COX-2 inhibition” means that COX-2 activity is inhibited to a greater extent than COX-1 activity.

“Treating” a condition refers to reversing, alleviating, inhibiting the progress of, or preventing the condition or one or more symptoms or signs of the condition.

A “COX-2 mediated condition” is any condition that responds favorably to COX-2 inhibition, particularly selective COX-2 inhibition.

A “condition for which COX-1 inhibition is disadvantageous” is a condition for which COX-1 inhibition exacerbates the condition (is contraindicated) or causes the condition to subside less quickly or completely than when COX-1 is not inhibited.

“Aliphatic” refers to substituted or unsubstituted alkanes, alkenes, alkynes, their cycloalkyl analogs, and combinations thereof.

“Aryl” refers to substituted or unsubstituted hydrocarbon groups forming aromatic rings, such as phenyl, naphthyl, pyrrolyl, pyridinyl, quinolinyl, and isoquinolinyl.

“Aryl-aliphatic” refers to any refers to an aryl group substituted by an aliphatic group, such as alkyl, for example a lower alkyl (also referred to as arylalkyl)

“Alkyl” refers to branched and straight chain hydrocarbons.

“Lower alkyl” refers to branched and straight chain hydrocarbons of from one to ten carbons inclusive, and is exemplified by such groups as propyl, isopropyl, butyl, 2-butyl, t-butyl, amyl, isoamyl, hexyl, heptyl, and octyl.

“Cycloalkyl” refers to cyclic alkanes, for example those having from one to ten carbons, such as cyclopropyl, cyclopentyl, cyclohexyl, cycloheptyl, and cyclooctyl.

A “biomolecule” is an organic molecule, whether naturally occurring, recombinantly produced, or chemically synthesized in whole or in part, that is, was or can be a part of a living organism. The term encompasses, for example, nucleotides, nucleosides, amino acids and monosaccharides, as well as oligomeric and polymeric species such as oligonucleotides and polynucleotides, peptidic molecules such as oligopeptides, polypeptides and proteins, saccharides such as disaccharides, oligosaccharides, polysaccharides, mucopolysaccharides and peptidoglycans (peptido-polysaccharides).

The term also encompasses, for example, ribosomes and enzyme cofactors. The amino acids include, for example, the twenty conventional amino acids (such as, lysine, argentine, and histadine), stereoisomers (for example, D-amino acids) of the conventional amino acids, unnatural amino acids such as, -disubstituted amino acids, N-alkyl amino acids, lactic acid, and other unconventional amino acids. Examples of unconventional amino acids include, but are not limited to, -alanine, naphthylalanine, 3-pyridylalanine, 4-hydroxyproline, O-phosphoserine, N-acetylserine, N-formylmethionine, 3-methylhistidine, 5-hydroxylysine, and nor-leucine. Peptidic molecules refer to peptides, peptide fragments, and proteins, that is, oligomers or polymers wherein the constituent monomers are amino acids linked through amide bonds. Nucleosides and nucleotides refer to nucleosides and nucleotides containing not only the conventional purine and pyrimidine bases, i.e., adenine (A), thymine (T), cytosine (C), guanine (G) and uracil (U), but also protected forms thereof, for example, where the base is protected with a protecting group such as acetyl, difluoroacetyl, trifluoroacetyl, isobutyryl or benzoyl, and purine and pyrimidine analogs. Common analogs include, but are not limited to, 1-methyladenine, 2-methyladenine, N (6)-methyladenine, N (6)-isopentyl-adenine, 2-methylthio-N (6)-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromo-guanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine. In addition, the terms nucleoside and nucleotide include those moieties that contain not only conventional ribose and deoxyribose sugars, but other sugars as well. Modified nucleosides or nucleotides also include modifications of the sugar moiety, for example, where one or more of the hydroxyl groups are replaced with halogen atoms or aliphatic groups, or are functionalized, for example, as ethers, or amines. Oligonucleotides include, for example, polydeoxynucleotides (containing 2-deoxy-D-ribose), polyribonucleotides (containing D-ribose), other types of polynucleotides which are an N-glycoside of a purine or pyrimidine base, and other polymers containing normucleotidic backbones, provided that the polymers contain nucleobases in a configuration that allows for base pairing and base stacking, such as is found in DNA and RNA. Thus, these terms include known types of oligonucleotide modifications, for example, substitution of one or more of the naturally occurring nucleotides with an analog, internucleotide modifications such as, for example, those with uncharged linkages (such as methyl phosphonates, phosphotriesters, and phosphoramidates, carbamates), with negatively charged linkages (such as phosphorothioates and phosphorodithioates), and with positively charged linkages (such as aminoalklyphosphoramidates and aminoalkylphosphotriesters), those containing pendant moieties, such as, for example, proteins (including nucleases, toxins, antibodies, signal peptides, and poly-L-lysine), those with intercalators (such as acridine and psoralen), and those containing chelators (for example, metals, such as radioactive metals, boron, and oxidative metals).

In certain cases, the biomolecule is a molecule that targets a particular type of tissue, for example a molecule that targets inflamed tissue, such as Very Late Antigen-4 (VLA4), which binds to Vascular Cell Adhesion Molecule-1 (VCAM1), which is expressed by endothelial cells at sites of inflammation, or a molecule that binds to selectins (such as P-, L-, and E-selectin), which also are expressed by endothelial cells at sites of inflammation. Selectin binding molecules, include, for example, sulfated disaccharides, as described in U.S. Pat. No. 5,977,080 to Rosen (such as lactose 6′-sulfate and lactose 3,6′-disulfate), sialylated and fucosylated oligosaccharides, and selectin binding glycoproteins, such as P-selectin glycoprotein ligand-1 (PSGL-1).

“Amine” or “amine group” refers to primary (NHR) or secondary (NR₂) groups wherein the R groups are organic groups such as aliphatic, aryl, or aryl-aliphatic substituted or unsubstituted hydrocarbons, NSAIDS, such as salicylic acid derivatives (for example, acetylsalicylic acid, diflunisal, salicylsalicylic acid), pyrazolon derivatives (for example, phenylbutazone, oxyphenbutazone, antipyrine and aminopyrine), para-aminophenol derivatives (for example, phenacetin and its active metabolite acetominaphin), propionic acid derivatives (for example, ibuprofen, naproxen, and flurbiprofen), and biomolecules, such as proteins, amino acids and nucleic acids.

“Substituted” refers to the attachment of one or more organic substituents to a particular group, such as attachment of an aliphatic, aryl, or aryl-aliphatic substituted or unsubstituted hydrocarbon, or an inorganic group such as a halogen group, for example I, Br, Cl, or F, or a nitro (NO₂) group.

“Unsubstituted” refers to a group that does not have additional substituents.

A “pharmaceutically acceptable cation” refers to any cation that does not render the compound unstable or toxic at contemplated dosages. Typically the cation is a group 1 or group 2 ion, such as sodium, potassium, calcium, and magnesium, for example, Na⁺, K⁺, Ca²⁺, and Mg²⁺.

Nitroxyl can be provided directly as HNO/NO⁻, but typically is provided with the use of a nitroxyl donor.

In some examples the nitroxyl donor is a nitroxyl-donating diazeniumdiolate. A diazeniumdiolate is a compound having the formula J-N(O)NO wherein J is any moiety. These compounds are generally known as diazeniumdiolates because they contain the N-oxy-N-nitroso (NONO) complex. Some diazeniumdiolates donate nitroxyl. These are referred to as nitroxyl-donating diazeniumdiolates. Such compounds include any compound where J is any moiety such that the compound donates nitroxyl. Examples of such compounds used in the disclosed methods have the formula: 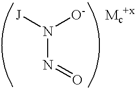
wherein J is oxide (O⁻), sulfite (SO₃⁻), amine, an NSAID, an aliphatic, aryl, or aryl-aliphatic substituted or unsubstituted hydrocarbon, or a biomolecule, and M_{c}^{+x} is a pharmaceutically acceptable cation, wherein x is the valence of the cation, and c is the smallest integer that results in a neutral compound. Examples of these compounds include Angeli's salt, where J is oxide, and sulfi/NO, where J is sulfite. In some specific cases J is alkyl, such as, lower alkyl, for example methyl, ethyl, n-propyl, isopropyl, n-butyl, isobutyl, secondary-butyl, tertiary butyl (t-butyl), cycloproyl, or cyclobutyl. In other cases J is aryl, for example phenyl. In certain cases nitroxyl-donating diazeniumdiolates include all the nitroxyl-donating diazeniumdiolates other than Angeli's salt and sulfi/NO.

Further examples of nitroxyl-donating diazeniumdiolates include diazeniumdiolates where J is an amine, for example a primary amine group (RNH) (a primary amine diazeniumdiolate). Examples of these compounds for use in the disclosed methods have the formula: 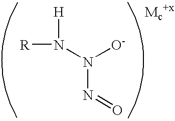
where R is an aliphatic, aryl, or aryl-aliphatic substituted or unsubstituted hydrocarbon, an NSAID, or a biomolecule, and M_{c}^{+x} is a pharmaceutically acceptable cation, wherein x is the valence of the cation, and c is the smallest integer that results in a neutral compound. In some instances R is alkyl, for example, lower alkyl, such as methyl, ethyl, n-propyl, isopropyl, n-butyl, isobutyl, secondary-butyl, tertiary butyl (t-butyl), cycloproyl, and cyclobutyl. In specific cases, R is isopropyl (IPA/NO) or t-butyl. In some cases R is aryl, for example phenyl. In still other cases R is aryl-aliphatic, where the aliphatic portion is alkyl, such as lower alkyl, for example ethylbenzene, n-propylbenzene, or isobutylbenzene. In specific cases, R is substituted with one or more inorganic groups, such as halogen groups, for example I, Br, Cl, or F, or nitro groups. For example, in some cases R is F substituted isopropyl, such as where R is (CH₃CH₂F)CH₂—)), (CH₂F)₂CH₂—)), (CHF₂)₂CH₂—)), or (CF₃)₂CH₂—)). In other specific cases R is an NSAID, for example, a salicylic acid derivative (for example, acetylsalicylic acid, diflunisal, salicylsalicylic acid), pyrazolon derivatives (for example, phenylbutazone, oxyphenbutazone, antipyrine and aminopyrine), a para-aminophenol derivative (for example, phenacetin and its active metabolite acetominaphin), or a propionic acid derivative (for example, ibuprofen, naproxen, and flurbiprofen).

In general, nitroxyl-donating diazeniumdiolates donate both nitroxyl and NO⁻. Nitroxyl versus NO⁻ donation by nitroxyl-donating diazeniumdiolates depends on the pH of the environment. The higher the pH the more likely the compound is to donate nitroxyl. Each nitroxyl-donating diazeniumdiolate donates nitroxyl at basic conditions (pH greater than 7, for example from a pH of greater than 7 to about 10). However, nitroxyl donation also occurs at acidic conditions (pH of less than 7) and neutral (pH of 7) conditions. For example, Angeli's salt donates nitroxyl at a pH of about 3 and greater, for example from a pH of about 3 to about 10. IPA/NO donates nitroxyl at a pH of about 5.5 and greater, for example from a pH of about 5.5 to about 10. For diazeniumdiolates, such as IPA/NO where J is a primary amine group (RNH), the nitroxyl donation pH is lower for compounds having larger R groups and/or with R groups having electron withdrawing groups such as halogen substituents. For example, the nitroxyl donation pH where R is t-butyl is lower than the nitroxyl donation pH where R is isopropyl. Also, the nitroxyl donation pH where R is isopropyl and has one or more halogen substituents, such as F, on one or more of the methyl branches, is lower than the nitroxyl donation pH where R simply is isopropyl. As human blood pH typically is about pH 7.3 to 7.4 the nitroxyl donation pH of the nitroxyl-donating diazeniumdiolates rarely will be of concern when such compounds are administered parenterally into the blood at normal physiologic pH.

However, pH may be of concern when the nitroxyl-donating diazeniumdiolate is injected directly into a site of inflammation or is taken orally. Sites of inflammation can be acidic, perhaps below the nitroxyl donation pH of a particular nitroxyl-donating diazeniumdiolate. Accordingly, a nitroxyl-donating diazeniumdiolate with a donation pH below the expected pH of the site to be treated is used. Such a compound is selected based on the discussion above concerning the nitroxyl donation pHs of various compounds and/or by testing the compound for its nitroxyl donation pH as discussed below. Alternatively, the nitroxyl-donating diazeniumdiolate is administered in a buffered solution, such as with phosphate buffered saline.

The stomach also typically is acidic, sometimes at a pH below the nitroxyl-donation pH of a particular nitroxyl-donating diazeniumdiolate. Accordingly, orally administered nitroxyl-donating diazeniumdiolates (and any other nitroxyl donors sensitive to pH) are administered in a form adapted to inhibit the nitroxyl-donating diazeniumdiolate from entering a subject's system until the compound has passed through the stomach. For example, the nitroxyl-donating diazeniumdiolate is administered in the form of an enterically coated tablet in some cases. Alternatively, the gastric pH can be increased by reducing or blocking the secretion of acid, for example by administration of a proton pump inhibitor.

In other cases the nitroxyl donor is a nitroxyl-donating S-nitrosothiol (RSNO), such as S-nitroso-L-cysteine ethyl ester, S-nitroso-L-cysteine, S-nitroso-glutathione, S-nitroso-N-acetyl-cysteine, S-nitroso-3-mercaptoethanol, S-nitroso-3-mercaptopropanoic acid, S-nitroso-2-aminonethanethiol, S-nitroso-N-acetyl penicillamine (SNAP), S-nitrosocaptopril. Wang et al., “New chemical and biological aspects of S-nitrosothiols,” Curr. Med. Chem., 7(8):821-34 (2000), describes nitroxyl formation from heterolytic decomposition of S-nitrosothiol compounds. In particular, S-nitrosoglutathione has been reported as capable of being reduced to nitroxyl in the presence of thiols. Hogg et al., Biochem. J, 323:477-481 (1997).

In other cases, the nitroxyl donor is a nitroxyl-donating hydroxamic acid (X(═O)NHOH) or its salt. For example, Piloty's acid (benzenesulfohydroxamic acid; (C₆H₅S(O)(O)NHOH)) is used as the nitroxyl donor. In some cases other hydroxamic acids that donate nitroxyl, such as other sulfohyrdroxamic acids and their derivatives are used as nitroxyl donors. In certain specific cases, the nitroxyl donor excludes Piloty's acid.

In still other cases, the nitroxyl donor is a nitroxyl-donating thionitrate having the formula (R—(S)—NO₂), wherein R is a polypeptide, an amino acid, a sugar, a modified or unmodified oligonucleotide, a straight or branched, saturated or unsaturated, aliphatic or aromatic, substituted or unsubstituted hydrocarbon. In particular cases, such compounds that form disulfide species are used as nitroxyl donors.

In other instances the nitroxyl donor is a nitroxyl-donating oxime having the formula (R₁R₂C═NOH) wherein R₁ and R₂ are, for example, hydrogen, or an aliphatic, aryl, or aryl-aliphatic substituted or unsubstituted hydrocarbon, for example where R₁ and R₂ are lower alkyl.

In some instances the nitroxyl donor is an analog and/or derivative of another nitroxyl donating compound, such as those described above. An analog is a molecule that differs in chemical structure from a parent compound, for example a homolog (differing by an increment in the chemical structure, such as a difference in the length of an alkyl chain), a molecular fragment, a structure that differs by one or more functional groups, or a change in ionization. Structural analogs are often found using quantitative structure activity relationships (QSAR), with technologies such as those disclosed in Remington: The Science and Practice of Pharmacology, 19^{th} Edition (1995), chapter 28. A derivative is a biologically active molecule derived from the base structure.

Any other nitroxyl donor can be used. One source helpful for determining nitroxyl donors is METHODS IN NITRIC OXIDE RESEARCH (Feelish M. & Stamler J. eds.) John Wiley & Sons, New York (1996).

Further, compounds are easily tested for nitroxyl donation with routine experiments. Although it is impractical to directly measure whether nitroxyl is donated, several tests are accepted for determining whether a compound donates nitroxyl. For example, the compound of interest can be placed in solution, for example in water, in a sealed container. After sufficient time for disassociation has elapsed, such as from several minutes to several hours, the headspace gas is withdrawn and analyzed to determine its composition, such as by gas chromatography and/or mass spectroscopy. If the gas primarily is N₂O, the test is positive for nitroxyl donation and the compound is a nitroxyl donor. Nitroxyl donation also can be detected by exposing the target donor to metmyoglobin (Mb³⁺). Nitroxyl reacts with Mb³⁺ to form an Mb²⁺—NO complex, which can be detected by changes in the ultraviolet/visible spectrum or by Electron Paramagnetic Resonance (EPR). The Mb²⁺—NO complex has a EPR signal centred around a g-value of about 2. Nitric oxide, on the other hand, reacts with Mb³⁺ to form an Mb³⁺—NO complex that is EPR silent. Accordingly, if the candidate compound reacts with Mb³⁺ to form a complex detectable by common methods such as ultraviolet/visible or EPR, then the test is positive for nitroxyl donatation.

Testing for nitroxyl donation in some cases is performed at a range of pHs to determine the nitroxyl donation pH of the nitroxyl-donating compound. For example, nitroxyl donation can be tested at a range of pHs such as 1, 2, 2.5, 3, 3.5, 4, 4.5, 5, 5.5, 6, 6.5, and so on. The lowest pH at which nitroxyl donation occurs is considered the nitroxyl donation pH. After the lowest pH at which nitroxyl donation occurs in a first set of tests is found, additional tests can be performed with narrower ranges of pH around the first determined nitroxyl donation pH to obtain a more specific nitroxyl donation pH. Alternatively, a nitroxyl donation test could be performed at an initial pH at which nitroxyl donation is known to occur while performing titration with acid to determine the pH at which nitroxyl donation ceases.

Compositions comprising more than one nitroxyl donating compound also are used in the disclosed methods. For example, IPA/NO and another compound that dissociates to generate nitroxyl, such as Angeli's salt, are used to inhibit COX-2 activity in some cases.

Nitroxyl donors are used to inhibit COX-2 activity. In particular, nitroxyl donors are used to selectively inhibit COX-2 activity over COX-1 activity. Nitroxyl donors in some cases have COX-2/COX-1 IC₅₀ ratios from about 0.25 to about 0.01 or less, for example, from about 0.25 to about 0.2, from about 0.2 to about 0.1, from about 0.1 to about 0.01, or less. In one particular example, the nitroxyl donor (Angeli's salt) has a COX-2/COX-1 IC₅₀ ratio of about 0.08.

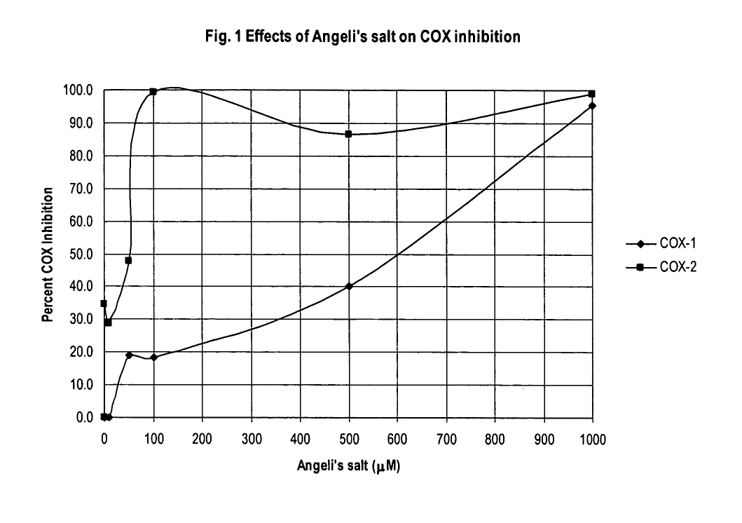

Nitroxyl inhibition of COX-2 and COX-1 is dose dependant. Of particular interest is that the dose response curve for COX-2 inhibition is significantly steeper than the dose response curve for COX-1 inhibition to about 100% COX-2 inhibition, as can be seen in FIG. 1. Accordingly, nitroxyl donors may be used at therapeutic doses that inhibit significantly more COX-2 activity than COX-1 activity. For example, nitroxyl donors are used to inhibit about 50% to about 100% of COX-2 activity, while inhibiting about 20% or less of COX-1 activity at the dose administered. In other instances nitroxyl donors are used to inhibit substantially all COX-2 activity while inhibiting COX-1 activity to a relatively low degree or inhibiting substantially only COX-2 activity at the dose administered. For example, nitroxyl donors are used to inhibit about 90% or more of COX-2 activity, for example about 100% of COX-2 activity, while inhibiting no more than about 50%, 40%, 30% or 20% of COX-1 activity at the administered dose.

Nitroxyl donors also are used to treat COX-2 mediated conditions. Examples of COX-2 mediated conditions include: pain, such as back or joint pain (such as that induced by arthritis or injury); headaches; inflammation; arthritis, such as osteoarthritis and rheumatoid arthritis; angiogensis; asthma; bronchitis; menstrual cramps and pain; premature labor; tendonitis; bursitis; fever; hepatitis; Parkinson's disease; Huntington's disease; skin-related conditions, such as, psoriasis, eczema, surface wounds, burns and dermatitis; post operative inflammation including from ophthalmic surgery, such as cataract surgery and refractive surgery; neoplasia, such as brain cancer, bone cancer, epithelial cell-derived neoplasia (epithelial carcinoma), such as basal cell carcinoma, adenocarcinoma, gastrointestinal cancer, such as lip cancer, mouth cancer, esophageal cancer, small bowel cancer and stomach cancer, colon cancer, liver cancer, bladder cancer, pancreatic cancer, ovarian cancer, cervical cancer, lung cancer, breast cancer and skin cancer, such as squamus cell and basal cell cancers, prostate cancer, renal cell carcinoma, and other known cancers that effect epithelial cells throughout the body, benign and cancerous tumors, growths, polyps, adenomatous polyps including, but not limited to, familial adenomatous polyposis and fibrosis resulting from radiation therapy; treatment of inflammatory processes in diseases, such as in vascular diseases, migraine headaches, periarteritis nodosa, thyroiditis, aplastic anemia, Hodgkin's disease, sclerodoma, rheumatic fever, diabetes including types I and II, neuromuscular junction disease including myasthenia gravis, white matter diseases including multiple sclerosis, sarcoidosis, nephrotic syndrome, Behcet's syndrome, polymyositis, gingivitis, nephritis, hypersensitivity, swelling occurring after injury, and myocardial ischemia; ophthalmic diseases and disorders, such as retinitis, retinopathies, uveitis, ocular photophobia, acute injury to the eye tissue, glaucoma, inflammation of the eye, and elevation of intraocular pressure; treatment of pulmonary inflammation, such as inflammation associated with viral infections and cystic fibrosis; central nervous system disorders, such as cortical dementias including Alzheimer's disease, vascular dementia, multi-infarct dementia, pre-senile dementia, alcoholic dementia, senile dementia, and central nervous system damage resulting from stroke, ischemia, and trauma; allergic rhinitis; respiratory distress syndrome; endotoxin shock syndrome; treatment of inflammations and/or microbial infections including, but not limited to, inflammations and/or infections of the eyes, ears, nose, throat, and/or skin; cardiovascular disorders, such as coronary artery disease, aneurysm, arteriosclerosis, atherosclerosis including atherosclerotic plaque rupture and cardiac transplant atherosclerosis, myocardial infarction, hypertension, ischemia, embolism, stroke, thrombosis, venous thrombosis, thromboembolism, thrombotic occlusion and reclusion, restenosis, angina, unstable angina, shock, heart failure, and coronary plaque inflammation; bacterial-induced inflammation, such as Chlamydia-induced inflammation, viral induced inflammation; inflammation associated with surgical procedures, such as vascular grafting, coronary artery bypass surgery, revascularization procedures, such as angioplasty, stent placement, endarterectomy, and vascular procedures involving arteries, veins, and capillaries; urinary and/or urological disorders, such as incontinence; endothelial dysfunctions, such as diseases accompanying these dysfunctions, endothelial damage from hypercholesterolemia, endothelial damage from hypoxia, endothelial damage from mechanical and chemical noxae, especially during and after drug, and mechanical reopening of stenosed vessels, for example, following percutaneous transluminal angiography (PTA) and percuntaneous transluminal coronary angiography (PTCA), endothelial damage in post-infarction phase, endothelium-mediated reocclusion following bypass surgery, and blood supply disturbances in peripheral arteries; disorders treated by the preservation of organs and tissues, such as organ transplants; disorders treated by the inhibition and/or prevention of activation, adhesion, and infiltration of neutrophils at the site of inflammation; immunodeficiency diseases, such as acquired immunodeficiency syndrome; and disorders treated by the inhibition and/or prevention of platelet aggregation. One of skill in the art would be able to identify these and other conditions that would respond favorably to COX-2 inhibition.

In some cases a subject with a COX-2 mediated condition is selected for administration of a nitroxyl donor. Such a subject is selected, for example, by making a diagnosis of any of the above conditions.

Nitroxyl donors further are used to selectively inhibit COX-2 activity in a subject having a condition for which COX-1 inhibition is disadvantageous, such as a condition for which COX-1 inhibition is contraindicated. Examples of these conditions include, for example, gastric mucosal disorders, such as gastrointestinal bleeding, peptic ulcers, gastritis, regional enteritis, ulcerative colitis, diverticulitis or a recurrent history of gastrointestinal lesions; with coagulation disorders, such as hypoprothrombinemia, thrombocytopenia, hemophilia, or other bleeding problems; and/or kidney disease.

In certain instances the subject is selected for administration of the nitroxyl donor. The subject could be selected, for example, by making a diagnosis of any condition for which COX-1 inhibition is disadvantageous.

Nitroxyl donors additionally are used to treat COX-2 mediated conditions in subjects having conditions for which COX-1 inhibition is disadvantageous, as described above. For example, the nitroxyl donor is used to treat conditions such as pain and/or arthritis in a subject with a gastric disorder. In certain cases a subject with a COX-2 mediated condition and a condition for which COX-1 inhibition is disadvantageous is selected for administration of the nitroxyl donor, for example, by making a diagnosis of a COX-2 mediated condition and a condition for which COX-1 inhibition is contraindicated.

In certain cases the nitroxyl donor is used to treat COX-2 mediated conditions in the absence of other NSAIDS, nitrosylated taxanes, other selective COX-2 inhibitors, histamine2-(H₂—) receptor antagonists, steroids, beta-receptor agonists, mast cell stabilizers, and phosphodiesterase (PDE) inhibitors. In particular cases, nitroxyl-donating diazeniumdiolates, such as Angeli's salt are used in the absence of such agents.

However, in other cases, the nitroxyl donor, such as a nitroxyl-donating diazeniumdiolate, for example a diazeniumdiolate having a primary amine group, such as IPA/NO, is administered to treat COX-2 mediated conditions with one or more other active ingredients, such as nitrosylated taxanes, other selective COX-2 inhibitors, such as celecoxib and rofecoxib, steroids, beta-receptor agonists, mast cell stabilizers, phosphodiesterase (PDE) inhibitors, other pain relievers including NSAIDS, such as acetaminophen or opiates such as morphine and vicodin; potentiators including caffeine; H2-antagonists including cimetidine, ranitidine, famotidine and nizatidine; decongestants including phenylephrine, phenylpropanolamine, pseudoephedrine, oxymetazoline, epinephrine, naphazoline, xylometazoline, propylhexedrine, or levodesoxyephedrine; anti-tussives including codeine, hydrocodone, caramiphen, carbetapentane, or dextromethorphan; and/or diuretics.

Typically, the nitroxyl donor (or combination of nitroxyl donors) is provided in the form of a pharmaceutical composition. A pharmaceutical composition comprising an effective amount of the nitroxyl donor as an active ingredient could easily be prepared by standard procedures well known in the art, with pharmaceutically acceptable non-toxic solvents and/or sterile carriers, if necessary. Such preparations are provided in a form for oral administration, such as an ingestible liquid or tablet, injection, or in any other administrable form. Typically the nitroxyl donor is provided in a form for parenteral administration. In cases where nitroxyl donating diazeniumdiolates are provided in a form for oral administration the pharmaceutical composition typically is enterically coated to protect the nitroxyl donor from gastric acid. However, this is not always necessary, for example if the nitroxyl donation pH of the compound is lower than the pH of the subject's stomach.

Enteric coating typically is accomplished by applying one or more enteric coating layers to a core composition covered with separating layer(s) by using a suitable coating technique. The enteric coating is designed to provide for transit of the drug through the acidic environment of the stomach into the less acidic intestine before dissolution of the composition and release of the active ingredient occurs. A suitable technique for enteric coating is described in U.S. Pat. No. 6,090,827. The enteric coating layer material generally is dispersed or dissolved in either water or in a suitable organic solvent. One or more polymers, separately or in combination, are used in some case as enteric coating layers, for example, solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). The enteric coating layers may contain pharmaceutically acceptable plasticizers to obtain desirable mechanical properties, such as flexibility and hardness of the enteric coating layers. Examples of these plasticizers include triacetin, citric acid esters, phthalic acid esters, dibutyl sebacate, cetyl alcohol, polyethylene glycols, polysorbates or other plasticizers. The amount of plasticizer is optimized for each enteric coating layer formula, in relation to selected enteric coating layer polymer(s), selected plasticizer(s) and the applied amount of said polymer(s). Additives such as dispersants, colorants, pigments, polymers, such as poly(ethylacrylate, methylmethacrylate), anti-tacking and anti-foaming agents are also included in the enteric coating layer(s) in some instances. Other compounds may be added to increase film thickness and to decrease diffusion of acidic gastric juices into the acidic susceptible active substance. To protect the acidic susceptible active substances, the enteric coating layer(s) typically has a thickness of approximately 10 μm or greater. The maximum thickness of the applied enteric coating layer(s) is normally only limited by processing conditions.

In some cases the nitroxyl donor (or combination of nitroxyl donors) is provided without a pharmaceutical carrier.

Nitroxyl can be administered in any manner. For example, nitroxyl can be administered orally, parenterally, or transdermally. Typically, the nitroxyl is administered parenterally. Administration can be by the subject, or by another, for example, a physician or nurse.

A therapeutically effective dose of the nitroxyl donor is used to inhibit COX-2 and treat COX-2 mediated condition. The therapeutically effective dose of the nitroxyl donor is a dose effective treat a COX-2 mediated condition or one or more symptoms or signs of such condition. Optimizing therapy to be effective across a broad population can be performed with a careful understanding of various factors to determine the appropriate therapeutic dose, in view of the inventors' disclosure that these agents cause selective inhibition of COX-2 activity.

In some examples the therapeutically effective dose is sufficient to achieve target tissue concentrations of nitroxyl or nitroxyl donors at levels that have been found to be sufficient to inhibit COX-2. Examples of such concentrations are found in Tables 1-3. Typically, such concentrations are from about 1 μM to about 500 μM, such as about 1 μM to about 100 μM, for example about 50-100 μM. Higher doses also are used in some cases.

Compounds also may be screened for COX-2 inhibition and selective COX-2 inhibition to determine therapeutic agents for COX-2 mediated conditions. Screening is accomplished by selecting a candidate compound and determining whether the candidate compound inhibits COX-2 and/or selectively inhibits COX-2. In some cases, candidate compounds are selected from compounds reported in the literature to donate nitroxyl. In other cases, candidate compounds are selected by testing a compound for nitroxyl donation. Candidate compounds also are selected from compounds with chemical structures similar to compounds known to donate nitroxyl. Tests for determining nitroxyl donation are described above. In some instances testing for nitroxyl donation includes determining the nitroxyl donation pH of the compound.

There are numerous methods for determining COX-2 inhibition (and COX-1 inhibition if determining selectivity). Several are discussed in Chan et al., J. Pharm. & Exp. Ther., 290:551-560 (1999). For example, the oxygen consumption of a COX inhibitor system (a COX system reacted with arachidonic acid in the presence of a nitroxyl donor) can be measured, such as with an oxygen electrode, and compared against the oxygen consumption of a control (for example, a standard or a control system with no inhibitory agent) wherein lower oxygen consumption indicates lesser COX activity. Another method includes measuring the oxidation of N,N,N′,N′-tetramethyl-p-phenylenediamine (TMPD) during the reduction of PGG₂ to PGH₂ in a COX inhibitor system by estimating the velocity of TMPD oxidation over a short period of time, such as from about 30 seconds to about 5 minutes, which is estimated by measuring the increase in absorbancy at about 590 nm to 610 nm. The velocity of oxidation of the inhibitor system is compared to the velocity of oxidation of a control, wherein a lower velocity indicates COX inhibition. A kit for performing this method is available from Immuno-Biological Laboratories. Another method includes measuring the inhibition of prostaglandin production in COX inhibitor systems and comparing the inhibition against a control, which can be COX-1 and COX-2 systems reacted with arachidonic acid in the absence of any inhibitory agent during the screening process or simply can be a standard for prostaglandin production in COX-1 and COX-2 systems.

In some cases, screening in the latter method (measuring prostaglandin production) is accomplished using an enzyme immunoassay (EIA) where COX systems include COX-1 or COX-2, an EIA reaction buffer, Heme, and either a control solution, such as NaOH, for control systems or inhibitor solutions of the nitroxyl donor in NaOH having a range of progressively increasing concentrations of the nitroxyl-donating compound. These systems are reacted with arachidonic acid for a period typically of about five to ten minutes. The COX reactions are stopped in each system, for example, by the addition of hydrochloric acid (HCl).

The prostaglandins (PGH₂) produced by the COX reaction in the control and inhibitor systems can be measured directly, but typically are converted to more stable PGF_{2α}, for example, by addition of stannous chloride. The relative amounts prostaglandins, such as PGF_{2α}, in each system are measured with an EIA kit. The EIA measures the amounts of prostaglandins in the systems based on binding to an assay antibody. Binding is determined by absorbance, for example, absorbance at 405 nm, with a plate reader, such as a Perkin Elmer plate reader. High binding results in low absorbance indicating low inhibition, while low binding results in high absorbance indicating high inhibition.

Once the inhibitor and control systems are prepared they are diluted in the EIA buffer at various dilution ratios, such as 1:1000, 1:2000, and 1:4000 and added to wells of the plate. The plate also contains prostaglandin (PG) standard systems, non-specific binding (NSB) systems, background COX systems, and zero binding (B₀) systems, which are used to calibrate the EIA. PG standard systems are prepared at various progressively increasing concentrations of PG, for example 15.6, 31.3, 62.5, 125, 250, 500, 1000, and 2000 PG/mL. Background COX systems contain either boiled COX-1 or COX-2 diluted in EIA reaction buffer. NSB, and B₀ systems include only the EIA buffer. Prostaglandin screening acetylcholinase tracer (reconstituted in the EIA buffer) is added to each system. Prostaglandin screening antiserum (reconstituted in the EIA buffer) is added to each system other than the NSB system. Typically, the plate is incubated from several hours to a day, such as from 4 to 24 hours at room temperature (about 22° C.). Ellman's reagent is added to each system in each well and the plate is agitated, such as on an orbital shaker, and protected from light, for example by covering with aluminum foil, for about an hour. The plate is then read on a plate reader, such as a Perkin Elmer plate reader.

An average value for the absorbance of the NSB systems is determined and this absorbance is used to correct the reading of other systems for NSB (by subtracting the average NSB absorbance). An average value for the absorbance of background COX also is determined and absorbance is later used to correct other systems for background COX levels.

An average value for B₀ is obtained and NSB corrected. Average values for absorbance of each PG standard system are obtained and NSB corrected. The percentage of prostanoid binding is determined by dividing the average NSB corrected absorbance (binding (B)) for each PG standard system by the average NSB corrected absorbance for B₀ systems (percentage equals B/B₀*100). A standard curve is prepared with % B/B₀ on the y axis and the prostaglandin concentration (PG/mL) on the x axis. Then the average % B/B₀ for the background, control, and inhibitor systems are determined for each concentration tested. The PG concentration for each of these systems is determined by finding the point on the standard curve that corresponds to the determined % B/B₀, determining the corresponding PG concentration on the x axis, and multiplying this concentration by the dilution factor used to prepare the system. To correct for background COX the PG concentration of the background COX-1 or COX-2 systems are subtracted from the COX-1 and COX-2 inhibitor and control systems, respectively. The percentage of inhibition is determined dividing the PG concentration for inhibitor systems by the PG concentration for control systems and multiplying by 100. At any particular concentration the nitroxyl-donating compound's selectivity can be assessed by comparing the percentage of COX-2 inhibition to the percentage of COX-1 inhibition. If the COX-2 inhibition percentage is greater than the COX-1 inhibition percentage, the nitroxyl-donating compound is a selective COX-2 inhibitor for that concentration. Typically, whether a nitroxyl-donating compound is a selective COX-2 inhibitor is determined by finding its COX-2/COX-1 IC₅₀ (where ratios below 1 indicate selectivity). This is generally accomplished by plotting COX-2 and COX-1 inhibition percentages for each concentration on a graph with percentage inhibition on the y axis and concentration on the x axis. The IC₅₀ for each type of inhibition is determined by finding the concentration on the graph at which the COX-type of interest is 50% inhibited. Then the COX-2/COX-1 IC₅₀ is determined. Of course, if only COX-2 inhibition is of interest, then COX-1 systems would not be used and simply the reduction in prostaglandin production in COX-2 inhibitor systems versus a control would be measured.

### EXAMPLE 1

This example demonstrates selective inhibition of COX-2 caused by the nitroxyl donor Angeli's salt.

Nitroxyl was investigated as an inhibitor of COX activity by measuring the inhibition of prostaglandin production when COX-1 and COX-2 systems were reacted with arachidonic acid either in the presence (inhibitor systems) or absence (control systems) of the nitroxyl donor Angeli's salt. The COX systems included 10 μL of either COX-1 or COX-2, 950 μL of an enzyme immunoassay (EIA) reaction buffer (0.1 M Tris-HCl at pH of about 8), 10 μL Heme, and either 20 μL of 10 mM NaOH for control systems or 20 μL solutions of Angeli's salt in 10 mM NaOH having concentrations of 0.001, 0.1, 1, 10, 50, 100, 500, and 1000 PM for inhibitor systems. Angeli's salt was maintained at a temperature of about 0° C. (kept on ice) until just prior to dilution in NaOH and use in testing COX inhibition. These systems were reacted with 10 μL of 10 mM arachidonic acid. After about 5 minutes the COX reactions were stopped in each system by the addition of about 50 μL of hydrochloric acid (HCl).

The PGH₂ produced by the COX reaction in the control and inhibitor systems was converted to the more stable PGF_{2α} by addition of stannous chloride. The relative amounts of PGF_{2α} in each system was measured with an EIA kit from Cayman Chemical (#560101). The EIA measured the amounts of PGF_{2α} in the systems based on binding to the assay antibody (Cayman anti-COX-1 or anti-COX-2), which was determined by absorbance at 405 nm with a Perkin Elmer plate reader as described above. The percentage of inhibition was determined by dividing the corrected amount of PGF_{2α} synthesized in the Angeli's salt systems by the corrected amount of PGF_{2α} synthesized in controls and multiplying by 100. Three dilutions (1:1000, 1:2000, and 1:4000) of each inhibitor system in the EIA buffer were prepared. Data is provided below only for the 1:2000 dilution as the 1:1000 dilution was too high for the sensitivity of the assay and the 1:4000 dilution was below the sensitivity of the assay.

Table 1 contains the data showing the percentages of COX-1 and COX-2 inhibition resulting from adding Angeli's salt to COX-1 and COX-2 systems to investigate inhibition of the COX reaction.

TABLE 1

| Angeli's salt | Percentage COX-1 | Percentage COX-2 |
|---|---|---|
| concentration μM | Inhibition | Inhibition |
| 0.000 | 0.0 | 0.0 |
| 0.001 | 0.0 | 0.0 |
| 0.100 | 0.0 | 0.0 |
| 1.0 | 0.0 | 34.6 |
| 10.0 | 0.0 | 28.8 |
| 50.0 | 19.1 | 47.9 |
| 100.0 | 18.3 | 99.2 |
| 500.0 | 40.2 | 86.4 |
| 1000.0 | 95.4 | 99.1 |

FIG. 1 is a graph showing the percentages of COX-1 and COX-2 inhibition caused by Angeli's salt. As can be seen, the COX-1 IC₅₀ for Angeli's salt is about 600 μM and the COX-2 IC₅₀ is about 50 μM. The COX-1/COX-2 IC₅₀ ratio of Angeli's salt is about 0.08. Angeli's salt caused a dramatic, dose-dependent inhibition in COX-2 activity for the range of concentrations from about 0.1 μM to about 100 μM at which concentration the COX-2 inhibition reached 100%. The percentage of COX-2 inhibition leveled off and even was reduced somewhat in the range above 100 μM to somewhat more than 500 μM. In the range above 500 μM to about 1000 μM the percentage of COX-2 inhibition returned to about 100%.

Further, Angeli's salt significantly inhibited COX-2 activity to a much greater degree than COX-1 activity at most concentrations below 1000 μM. For example, at a concentration of 0.1 μM Angeli's salt inhibited about 35% of COX-2 activity while COX-1 was not inhibited to a measurable degree. Further, at a concentration of about 50 μM Angeli's salt inhibited about 50% of COX-2 activity while inhibiting only about 19% of COX-1 activity. At concentrations from about 50 μM to about 100 μM Angeli's salt inhibited from about 50% to about 100% of COX-2 activity while inhibiting no more about 19% of COX-1 activity. Interestingly, the inhibition of COX-2 by Angeli's salt increased sharply in a dose dependent fashion over this range while the inhibition of COX-1 did not increase. These data demonstrate that a nitroxyl donor, such as Angeli's salt, can inhibit substantially all COX-2 activity, for example from about 90% to about 100%, while inhibiting COX-1 activity to a relatively low degree, for example, about 20% or less, or inhibiting substantially only COX-2.

### EXAMPLE 2

The same process for testing COX inhibition was used as above in Example 1. Table 2 contains the data showing the percentages of COX-1 and COX-2 inhibition resulting from adding Angeli's salt to COX-1 and COX-2 systems to investigate inhibition of the COX reaction.

TABLE 2

| Angeli's salt | Percentage COX-1 | Percentage COX-2 |
|---|---|---|
| concentration μM | Inhibition | Inhibition |
| 0.000 | 0 | 0 |
| 0.001 | 97.5 | n/a |
| 0.100 | 24.7 | 98.9 |
| 1.0 | 57.5 | n/a |
| 10.0 | n/a | n/a |
| 50.0 | 16.4 | n/a |
| 100.0 | 45.7 | 98 |
| 500.0 | 62.5 | 105.7 |
| 1000.0 | 68.6 | 106 |

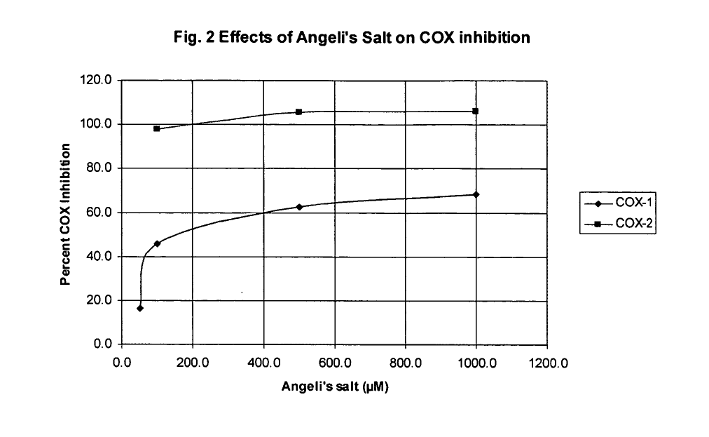

Due to the sensitivity of the EIA kits used to determine COX inhibition the data for several concentrations were indeterminable. Further, at concentrations below about 50 μM this assay resulted in unreliable data. Thus, these data could not be used to reliably determine a COX-2/COX-1 IC₅₀ ratio. However, a reasonable estimate of the COX-2 IC₅₀ is about 50 μM with a COX-1 IC₅₀ of about 200-250 μM resulting in a COX-2/COX-1 IC₅₀ ratio of from about 0.25 to about 0.2. The performance of the assay kit in this example suggests that the data obtained in this example might contain significant errors. Nevertheless, as can be seen in FIG. 2, the general trend observable from these data demonstrates selective inhibition of COX-2 over COX-1.

### EXAMPLE 3

In this example the COX-2 inhibitory effect of the nitroxyl donor IPA/NO was compared to the inhibition by Angeli's salt. IPA/NO has a similar half-life to Angeli's salt so it is a good compound to use to compare to Angeli's salt. The same process for testing COX inhibition was used as above in Example 1, however, in this case nitroxyl donors and controls were used at concentrations of 25, 50, 75, 100, and 1000 μM and only COX-2 inhibition was determined.

Table 3 contains data showing the percentages of COX-2 inhibition resulting from adding either Angeli's salt or IPA/NO to COX systems to inhibit the COX reaction.

TABLE 3

| | |
|---|---|
| | Percentage |
| Nitroxyl donor | COX-2 inhibition |

| concentration μM | Angeli's salt | IPA/NO |
|---|---|---|
| 25 | 5.4 | 6.7 |
| 50 | 13.7 | 59.5 |
| 75 | 49.2 | 46.4 |
| 100 | 59.9 | 62.2 |
| 1000 | 60.4 | 86.8 |

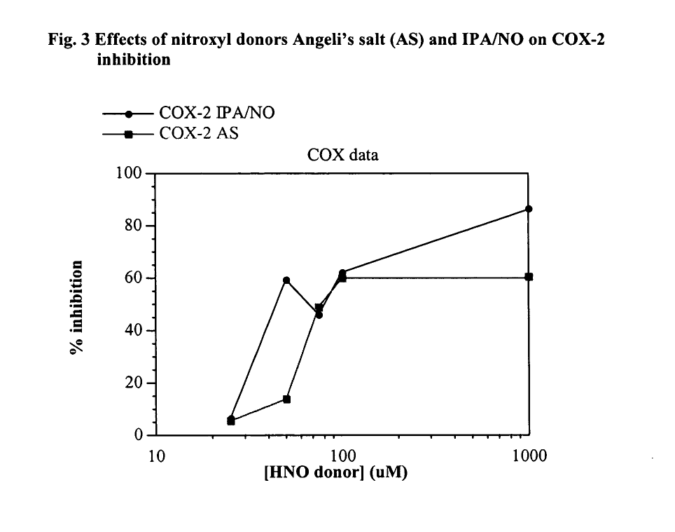

As shown in FIG. 3, IPA/NO and Angeli's salt exhibited similar COX-2 inhibition at a concentration of about 25 μM. However, in the range from about 25 μM to about 50 μM IPA/NO inhibited COX-2 to a significantly greater degree than Angeli's salt. Between the concentrations of about 50 μM to about 100 μM the percentages of COX-2 inhibition caused by IPA/NO and Angeli's salt converged. However, at concentrations over about 100 μM IPA/NO inhibited COX-2 to a greater degree than Angeli's salt. The overall trend shown by these data demonstrates that IPA/NO is a more effective inhibitor of COX-2 than Angeli's salt.

The above-described examples merely describe particular embodiments of the disclosed methods. They are not intended to be limiting in any way. Moreover, although embodiments of the methods provided have been described herein in detail, it will be understood by those of skill in the art that variations may be made thereto without departing from the spirit and scope of the appended claims.

1. A method for treating a cyclooxygenase-2 mediated condition, comprising:
administering to a subject having a cyclooxygenase-2 mediated condition a therapeutically effective dose of a nitroxyl-donating diazeniumdiolate other than Angeli's salt or sulfi/NO, wherein the nitroxyl-donating diazeniumdiolate is administered under conditions that cause it to donate nitroxyl, and wherein the dose is effective to inhibit cyclooxygenase-2 activity and to treat the cyclooxygenase-2 mediated condition.

2. The method of claim 2, wherein the nitroxyl-donating diazeniumdiolate has the formula 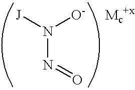
wherein j is amine, an aliphatic, aryl, or aryl-aliphatic substituted or unsubstituted hydrocarbon, or a biomolecule and M_{c}^{+x} is a pharmaceutically acceptable cation, wherein x is the valence of the cation, and c is the smallest integer that results in a neutral compound.

3. The method of claim 2, wherein J is lower alkyl.

4. The method of claim 2, wherein J is amine.

5. The method of claim 4, wherein J is primary amine.

6. The method of claim 5, wherein the nitroxyl-donating diazeniumdiolate has the formula 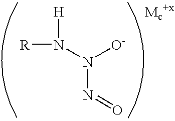
where R is an aliphatic, aryl, or aryl-aliphatic substituted or unsubstituted hydrocarbon, an NSAID, or a biomolecule and M_{c}^{+x} is a pharmaceutically acceptable cation, wherein x is the valence of the cation, and c is the smallest integer that results in a neutral compound.

7. The method of claim 6, wherein R is alkyl.

8. The method of claim 7, wherein R is lower alkyl.

9. The method of claim 8, wherein R is isopropyl.

10. The method of claim 1, wherein the nitroxyl-donating diazeniumdiolate is administered in the form of an enterically coated pharmaceutical composition.

11. The method of claim 1, wherein the cyclooxygenase-2 mediated condition is selected from the group consisting of pain, headache, arthritis, cancer, and Alzheimer's disease.

12. The method of claim 1, wherein cyclooxygenase-2 activity is inhibited selectively over cyclooxygenase-1 activity at the therapeutically effective dose.

13. The method of claim 12, wherein from about 50% to about 100% of cyclooxygenase-2 activity is inhibited and no more than about 20% of cyclooxygenase-1 activity is inhibited at the therapeutically effective dose.

14. The method of claim 13, wherein from about 90% to about 100% of cyclooxygenase-2 activity is inhibited at the therapeutically effective dose.

15. The method of claim 12, wherein the therapeutically effective dose is in an amount of the nitroxyl-donating diazeniumdiolate sufficient to achieve a concentration of the nitroxyl-donating diazeniumdiolate of about 50-100 μM in a target tissue in the subject.

16. A method for treating a cyclooxygenase-2 mediated condition in a subject having a condition for which cyclooxygenase-1 inhibition is disadvantageous, comprising:
administering to a subject having a cyclooxygenase-2 mediated condition and a condition for which cyclooxygenase-1 inhibition is disadvantageous a therapeutically effective dose of a nitroxyl-donating compound, wherein the nitroxyl-donating compound is administered under conditions that cause it to donate nitroxyl, and wherein the dose is effective to selectively inhibit cyclooxygenase-2 activity and to treat the cyclooxygenase-2 mediated condition.

17. The method of claim 16, wherein the nitroxyl-donating compound is administered in the absence of other NSAIDS, nitrosylated taxanes, other selective COX-2 inhibitors, histamine2-receptor antagonists, steroids, beta-receptor agonists, mast cell stabilizers, and phosphodiesterase inhibitors.

18. The method of claim 16, further comprising selecting a subject having a cyclooxygenase-2 mediated condition and a condition for which cyclooxygenase-1 inhibition is disadvantageous for administration of the nitroxyl-donating compound.

19. The method of claim 16, wherein the nitroxyl-donating compound is a nitroxyl-donating diazeniumdiolate.

20. The method of claim 19, wherein the nitroxyl-donating diazeniumdiolate has the formula 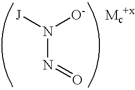
wherein J is oxide, sulfite, amine, an aliphatic, aryl, or aryl-aliphatic substituted or unsubstituted hydrocarbon, an NSAID, or a biomolecule and M_{c}^{+x} is a pharmaceutically acceptable cation, wherein x is the valence of the cation, and c is the smallest integer that results in a neutral compound.

21. The method of claim 20, wherein J is lower alkyl.

22. The method of claim 20, wherein J is amine.

23. The method of claim 22, wherein the nitroxyl-donating diazeniumdiolate has the formula 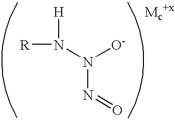
where R is an aliphatic, aryl, or aryl-aliphatic substituted or unsubstituted hydrocarbon, an NSAID, or a biomolecule and M_{c}^{+x} is a pharmaceutically acceptable cation, wherein x is the valence of the cation, and c is the smallest integer that results in a neutral compound.

24. The method of claim 23, wherein R is alkyl.

25. The method of claim 24, wherein R is lower alkyl.

26. The method of claim 25, wherein R is isopropyl.

27. The method of claim 19, wherein the nitroxyl-donating diazeniumdiolate is administered in the form of an enterically coated pharmaceutical composition.

28. The method of claim 16, wherein the nitroxyl-donating compound is a nitroxyl-donating hydroxamic acid.

29. The method of claim 28, wherein the nitroxyl-donating hydroxamic acid is Piloty's acid.

30. The method of claim 16, wherein the nitroxyl-donating compound is a nitroxyl-donating S-nitrosothiol.

31. The method of claim 30, wherein the nitroxyl-donating S-nitrosothiol is S-nitroso-glutathione.

32. The method of claim 16, wherein from about 50% to about 100% of cyclooxygenase-2 activity is inhibited and no more than about 20% of cyclooxygenase-1 activity is inhibited at the therapeutically effective dose.

33. The method of claim 32, wherein from about 90% to about 100% of cyclooxygenase-2 activity is inhibited at the therapeutically effective dose.

34. The method of claim 16, wherein the cyclooxygenase-2 mediated condition is selected from the group consisting of pain, headaches, arthritis, cancer, and Alzheimer's disease.

35. The method of claim 16, wherein the condition for which cyclooxygenase-1 inhibition is disadvantageous is selected from the group consisting of gastric mucosal disorders, coagulation disorders, and kidney disorders.

36. The method of claim 35, wherein the condition for which cyclooxygenase-1 inhibition is disadvantageous is a gastric mucosal disorder.

37. The method of claim 36, wherein the gastric mucosal disorder is selected from the group consisting of gastrointestinal bleeding, peptic ulcers, gastritis, regional enteritis, ulcerative colitis, and diverticulitis.

38. A method for screening compounds for cyclooxygenase-2 inhibition, comprising:
selecting a candidate compound; and
determining whether the candidate compound inhibits cyclooxygenase-2.

39. The method of claim 38, wherein selecting a candidate compound comprises selecting a compound known to donate nitroxyl.

40. The method of claim 39, wherein selecting a compound known to donate nitroxyl comprises testing a compound for nitroxyl donation, wherein the compound is known to donate nitroxyl if the compound tests positive for nitroxyl donation.

41. The method of claim 40, wherein testing for nitroxyl donation includes testing for nitroxyl donation at a range of pHs.

42. The method of claim 38, wherein the candidate compound is a primary amine diazeniumdiolate.

43. The method of claim 38, further comprising determining whether the candidate compound selectively inhibits cyclooxygenase-2.

44. The method of claim 43, wherein determining whether the candidate compound selectively inhibits cyclooxygenase-2 comprises determining the cyclooxygenase-2/cyclooxygenase-1 IC₅₀ ratio of the candidate compound, and wherein cyclooxygenase-2 is selectively inhibited over cyclooxygenase-1 if the cyclooxygenase-2/cyclooxygenase-1 IC₅₀ ratio is less than 1.

45. The method of claim 43, wherein the determining whether the candidate compound selectively inhibits cyclooxygenase-2 comprises:
reacting a cyclooxygenase-1 and a cyclooxygenase-2 system with arachidonic acid in the presence of the candidate compound;
measuring prostaglandin production in the cyclooxygenase-1 and the cyclooxygenase-2 systems; and
comparing the measured prostaglandin production in the cyclooxygenase-1 and the cyclooxygenase-2 systems against a control for each system.
